(19) United States
(12) Patent Application Publication
(10) Pub. No.: US 2014/0350492 A1
(43) Pub. Date: Nov. 27, 2014

(54) EYEDROP DISPENSER

(71) Applicants: Ever Rojas Escalante, Barranquilla (CO); Fabián Ramos Archibold, Barranquilla (CO); Elena Ibarreche Fonseca, Bogota (CO)

(72) Inventors: Ever Rojas Escalante, Barranquilla (CO); Fabián Ramos Archibold, Barranquilla (CO); Elena Ibarreche Fonseca, Bogota (CO)

(21) Appl. No.: 13/902,824

(22) Filed: May 26, 2013

(51) Int. Cl. A61F 9/00; B65D 23/10

(52) U.S. Cl. CPC A61F 9/0008; B65D 23/102
USPC 604/295; 215/384

(57) ABSTRACT

The present invention relates to a dispenser comprising: a container defining a reservoir, said container having a resilient sidewall, a closed end, and a dispensing tip opposite the closed end, said dispensing tip including a passageway in fluid communication with the reservoir, and said resilient side wall including at least one finger indent; and a lid comprising a tube configured to cover the dispensing tip and an outer wall configured to align with the resilient side wall when the tube covers the dispensing tip, the outer wall including at least one finger indent;wherein the lid is removably connectable to the container by a threaded connection between the tube and the dispensing tip.

This application claims the priority benefit under 35 U.S.C. section 119 of U.S. Provisional Patent Application No. 61/651,613 entitled “Eyedrop Dispenser” filed on May 25, 2012; which is in its entirety herein incorporated by reference.

### FIELD OF THE INVENTION

The present invention is directed to ocular treatment apparatus and, in particular, to ocular treatment apparatus that can apply eye drops of liquid medicament safely and easily.

The instant invention relates generally to dispensing devices and more specifically it relates to an improved eye drop applicator.

This invention generally relates to devices which assist the application of medicated solutions to the eye without the aid of another person. This invention pertains to the field of eye drop dispensing devices. More particularly, it relates to the field of those eye drop dispensing devices which allow for the proper positioning of the dispensing device over the eye with the use of one hand so as to provide for the safe and easy application of the ocular medicament.

This invention also relates to dispensing liquids to human eyes. The present invention also relates to liquid dispensers, and more particularly to a dispenser which is adapted to apply eye drop solutions or medications to the eye.

The present invention further pertains to the art of liquid dispensers, and more particularly to a manually held drop dispenser for accurately and easily dispensing small amounts of liquids at confined drop-releasing locations. The present invention is particularly applicable to an eye drop dispenser and will be described with reference thereto.

The present invention also relates generally to devices for dispensing drops and particularly, but not by way of limitation, to an apparatus for administering uniformly measured drops into the eye.

### BACKGROUND OF INVENTION

There is a growing trend in the ophthalmology community to seek safer, more efficient and convenient methods for dispensing ophthalmic solutions, in recent years there has been many attempts to resolve this issue.

Human eyes frequently need to have a medicine, water, or a buffered fluid administered to them. For example, lubricating fluid is needed when an eye is covered by a contact lens for an extended time; and medicinal solutions are required for treatment of infections or diseases, such as glaucoma. It is also necessary to irrigate eyes with a saline solution when they have been contacted with a harmful agent. For example, in some laboratory accidents, an acid or alkali may splash into an eye, and must immediately be diluted and washed away.

Illnesses of the eye, such as glaucoma, keratoconjunctivitis (“dry eyes”), infections of the eye, and other conditions are often treated with eye drops. The elderly and the infirm often have difficulty administering eye drops to themselves. This results in a significant amount of often expensive eye medication being spilled during each application.

The self-administering process involves raising an eye drop container above one's eye, taking aim over the eye, and squeezing the container a certain number of times (as prescribed) to dispense the eye drop solution. If the first drop misses the eye, the person must adjust their position to take better aim for the second drop. This results in wasted medication. Moreover, if the user does not realize that the first drop misses or becomes frustrated with repeated misses, inadequate treatment and significant waste of medication results.

Conventionally, drops are applied to the eye with an eye dropper generally in the form of a glass tube having a rubber bulb at one end. This manner of application has a number of disadvantages such as discomfort from anticipation of the drop and involuntary blinking as the drop is seen falling into the eye, which may then result in the drop actually falling on the lids. Commonly an excess of fluid in the drop runs onto the skin about the eye and this is not only inconvenient but also gives rise to the possibility of systemic poisoning by absorption of particular fluids through the skin. Furthermore, and particularly applicable to self-administration of eye drops, is the problem of uncertain dosage originally applied and the indeterminate fraction thereof that is squeezed out of the eye by reflex blinking

The use of eye dropper bottles, for example soft-sided squeeze bottles, having a nozzle with a discharge opening for applying drops of medication or cleansing fluids directly into the eye is conventional and well-known. It is also well known that considerable difficulty is often encountered in trying to hold the hand steady enough to ensure that the liquid from the eye dropper bottle drops into the correct place in the eye and, with squeeze bottles, the bottle often moves sufficiently when squeezed to cause the liquid to miss the eye and fall onto the face, which is both irritating and wasteful. A further problem which is often encountered with the use of eye dropper bottles is that when the user is looking directly upward at the end of the dropper, the distance of the lower end of the dropper from the eye cannot be accurately determined, and this gives rise to the danger of the eyeball being contacted by the dropper, thereby increasing the risk of damage and infection to the eye. The above problems are especially acute with users who are elderly, nervous, suffer from hand tremors, or who generally lack adequate coordination.

Aside from the occasional administration of eye drops as may occur with many people, there is also the necessity of continued use of eye drops in certain instances. Thus, for example, in the treatment of glaucoma it is conventional to prescribe four drops of pilocarpine per day for life. The necessity of continued usage of eye drops as in the foregoing instance usually requires the drops to be self-administered and the present invention is particularly adapted to this application.

The conventional eye drop container is a small, cylindrical bottle having nozzle. Eye drops are dispensed into the eye by positioning the bottle in a mostly inverted position over the eye, and gently compressing the bottle in order to force medicative solution through the nozzle and into the eye. This procedure requires the user to first tilt his or her head back into a substantially horizontal position. The user must then retract one or both eyelids from the eye with one hand, while dispensing eye drops with the other.

For most people, the procedure for dispensing eye drops is difficult and imprecise. With the conventional dispenser, the user attempts to drop medicament directly onto the surface of the eyeball. This produces unwanted blinking and natural tear flow due to the particular sensitivity of the conjunctiva. This, in turn, limits the time in which the medicament remains effective.

The preferred area of the eye for dispensing is the lower eyelid, or conjunctival sac. Yet, the use of a conventional eye drop container makes it difficult to align the nozzle with the conjunctival sac.

An additional obstacle to the proper dispensing of eye drops is the risk of contamination to the medicament. With the conventional eye drop dispenser, the user is required to place the fingers of one hand in close proximity to the eye so as to retract the upper and/or lower eyelids from the surface of the eyeball. This creates a risk of contamination of the medicament, as well as infection of the eye.

Additionally, the increase in smog and other impurities in the air in most urban areas coupled with the presence of various types of allergies such as pollen and dust has resulted in the increased need for periodically treating the eyes with various ophthalmic products designed to relieve the discomforts caused by these impurities. In addition, changing consumer life styles have placed an additional strain on eyes; often requiring some kind of relief. For example, activities such as extended viewing of television, driving, close work such as sewing and needlework cause eye strain while outdoor activities where sun and/or wind are involved such as skiing, tennis, golf and sailing cause eye discomfort while activites such as swimming, surfing and smoking are considered more eye irritating. All of the foregoing place an added strain on normal functioning eyes and oftentimes require some kind of relief. Today, there are many over-the-counter ophthalmic products available for temporary relief of these irritations.

In view of the foregoing it is no surprise that recently there has been a substantial growth in the eye hygiene category; particularly in the cosmetic products available. These products have advertising positionings that emphasize “get-the-red-out”, soothe and/or cleanse the eyes. Additionally, prescription products available for treating various conditions ranging from infection to diseases have increased significantly.

In the treatment of eyes with either cosmetic products or prescriptive preparations it is imperative that the product dispensed into the eye or occular cavity be sterile. With today's advanced production technology, the production and filling of sterile, ophthalmic products is fairly straight foward. With the sophisticated quality control techniques available today it is most unlikely that a contaminated product would become available to the consumer. However, after these products are filled, distributed and purchased by the consumer there is a substantial risk of their being contaminated during use. For example, in many dispensers available today contamination can result from contaminants in the air being drawn into the dispenser after some product has been dispensed. Such contaminants can then come into contact with the product remaining in the dispenser. This exchange of potentially contaminated air for the product dispensed is one of the factors that has necessitated the use of various preservative additives found in most ophthalmic products today.

Accordingly there is a long felt need in to art for improved dispensers for providing eyedrops to the ophthalmic cavity.

### OBJECTS OF THE INVENTION

A primary object of the present invention is to provide an improved eye drop applicator that will overcome the shortcomings of the prior art devices.

It is another object of the present invention to provide a plastic dropper bottle that will enable its users to dispense ophthalmic solutions in either eye with a steady hand, independently.

An object of the present invention is to provide a relatively simple eyedrop dispenser which is easy to use, i.e. which can be used with one hand while accurately instilling drops in an eye.

Still another object of the present invention is to provide a plastic dropper bottle that will enable its users to dispense ophthalmic solutions in either eye without touching the eye.

Another object of the present invention to provide a method for dispensing a sterile ophthalmic product into the occular cavity.

A further object is to provide an improved eye drop applicator that is simple and easy to use.

A still further object is to provide an improved eye drop applicator that is economical in cost to manufacture.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE FIGURES

FIG. 1 is a side view of the container defining the reservoir of the invention useful for making the dispensing device of the invention.

FIG. 2 is a side view of the dispensing device of the invention including the dispensing tip and lid of the device.

FIG. 3 is a view of the dispensing device illustrating the finger indent on the outside wall of the lid.

FIG. 4 is a front view of the dispensing device illustrating the finger indent on the resilient sidewall of the container.

FIGS. 5A and 5B are views of the lid of the invention illustrating the internal tube and finger indents respectively.

FIG. 5C is a top view of the lid of the invention showing the finger indents.

FIG. 6 shows two photographs of the dispensing device of the invention with the lid in place showing the finger indent on the lid and the finger indent on the sidewall.

It should be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted. It should be understood, of course, that the invention is not necessarily limited to the particular embodiments illustrated herein.

### SUMMARY OF THE INVENTION

The invention is principally directed to a disposable, integrally formed, thermoplastic dispenser suitable primarily for self-applied unit-dose or limited-dose dispensing of a liquid medicament to the eye.

The invention provides a container useful for making a dispensing device, said container having a resilient sidewall, a closed end, said resilient side wall including at least one finger indent, a threaded neck portion, and another resilient side wall opposite the resilient sidewall having at least one finger indent.

In accordance with the present invention, there is also provided a dispenser comprising: a container defining a reservoir, said container having a resilient sidewall, a closed end, and a dispensing tip opposite the closed end, said dispensing tip including a passageway in fluid communication with the reservoir, and said resilient side wall including at least one finger indent; and a lid comprising a tube configured to cover the dispensing tip and an outer wall configured to align with the resilient side wall when the tube covers the dispensing tip, the outer wall including at least one finger indent; wherein the lid is removably connectable to the container by a threaded connection between the tube and the dispensing tip.

There is also provided a container unit which is made of a bottom portion, a hollow trunk portion which is linked to its periphery, a cylindrical neck portion which continues a shoulder portion of the trunk portion, one side of the trunk portion having at least one finger indentation and a threaded portion on the cylindrical neck portion.

The present invention is also directed to a dispenser which can be easily gripped in a positive manner and thereafter manoeuvred with a high degree of confidence that the grip on the dispenser will be maintained. Further, the gripping means for this dispenser is such that the dispenser can be gripped through the finger indentitation in the resilient wall of the dispenser. This serves to increase the degree of confidence of the person gripping the dispenser.

### DETAILED DESCRIPTION OF THE INVENTION

The fluid dispensing device of the present invention is intended for delivery of sterile fluids such as therapeutic ophthalmic fluids intended for administration to the eye, or sterile fluids intended for administration to any bodily surface. The fluid dispensing device of the present invention has dispensing means so that fluids can be dispensed without the dispensing means contacting the surface to which the fluids are administered.

The fluid dispensing means of the present invention consists of a container 2 which can be formed from a variety of materials such as, for example, soft or hard plastic, or other manually deformable material, or glass. The container, if made of plastic, can be produced by conventional blow molding technique. It is to be understood that the container of the present invention is not limited to any specific material or to its manufacture by any specific process as it will be understood by those skilled in the art that many different materials and various manufacturing techniques can be employed.

The container 2 is adapted to contain a liquid medication, preferably a physiologically acceptable, liquid ophthalmic medication. The upper portion of the container 2 has a threaded neck to provide a dispenser. The bottle 2 may be fitted with a lid. The dispensing tip has an internal channel communicating with the bottle and is calibrated to deliver a predetermined amount of liquid.

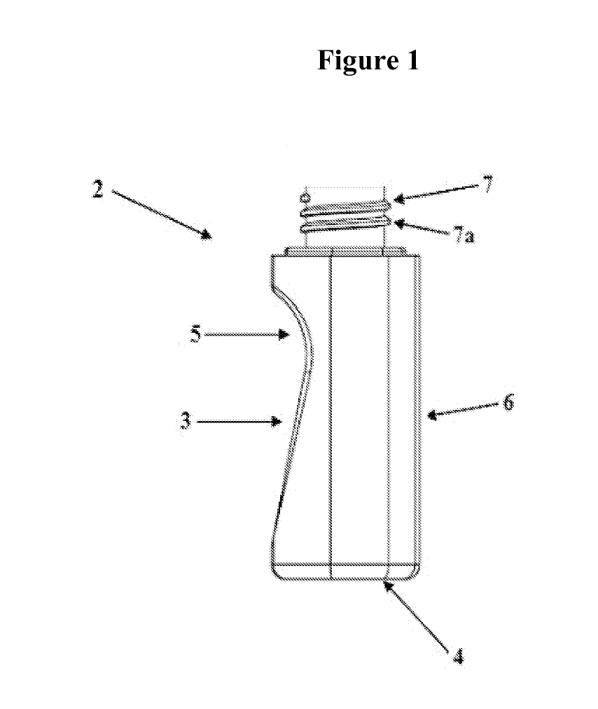

In a first embodiment of the invention as shown in FIG. 1, there is provided a container 2 useful for making a dispensing device, said container having a resilient sidewall 3, a closed end 4, said resilient side wall including at least one finger indent 5, a threaded neck portion 7 and 7a, and another resilient side wall 6 opposite the resilient sidewall 3 which has at least one finger indent 5.

The container can be made in various sizes and is designed to store the ophthalmic product. Generally, the cavity or reservoir means will contain from between about 5 and about 150 cc of the product.

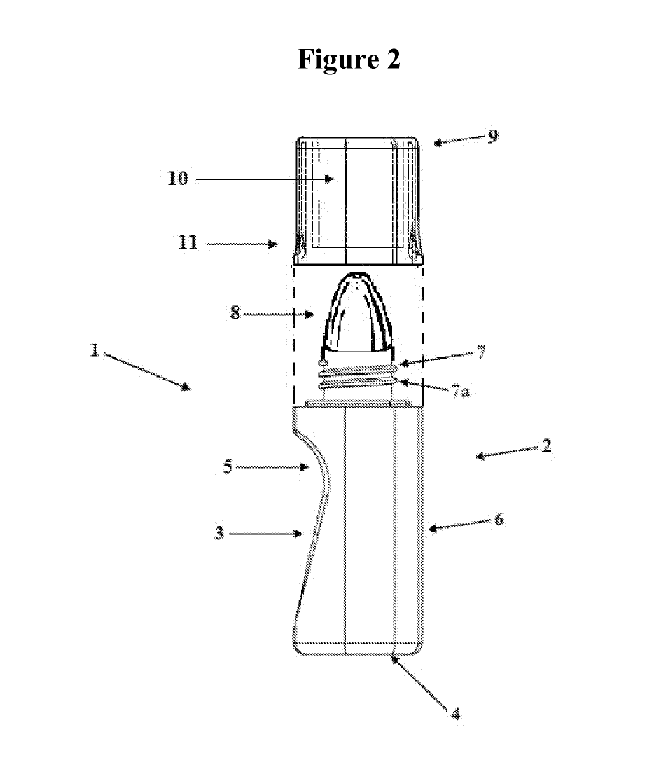

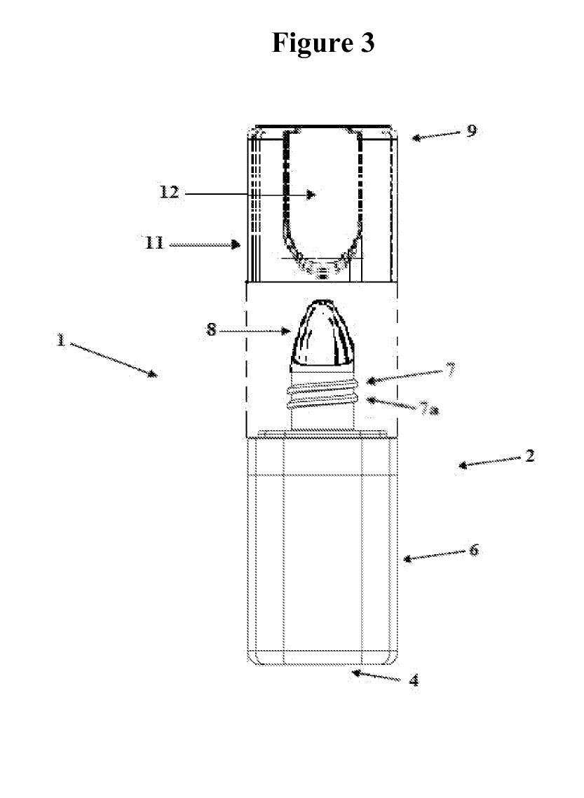

In a second embodiment shown in FIG. 2, the invention is a dispenser 1 comprising a container 2 defining a reservoir, said container having a resilient sidewall 3, a closed end 4, and a dispensing tip 8 opposite the closed end, said dispensing tip including a passageway in fluid communication with the reservoir, and said resilient side wall including at least one finger indent 5; and a lid 9 (shown on top of the dispensing tip) comprising a tube 10 configured to cover the dispensing tip and an outer wall 11, configured to align with the resilient side wall when the tube covers the dispensing tip, the outer wall 11 including at least one finger indent (shown in the view of FIG. 3); wherein the lid is removably connectable to the container by a threaded connections 7 and 7a between the tube and the dispensing tip.

The eyedropper dispenser of the present invention which is used principally for medical use, is made from flexible thermoplastic. It is formed by blow molding or vacuum molding into which is simultaneously filled a predetermined volume of pharmaceutical fluid, and a lid, removably screwed onto a male thread which is formed on a threaded cylindrical portion of the container unit.

Referring to FIG. 3, the invention provides a dispensing device as explained in detail in connection with FIG. 2, where the lid shows the finger indent 12 on the outerwall 11.

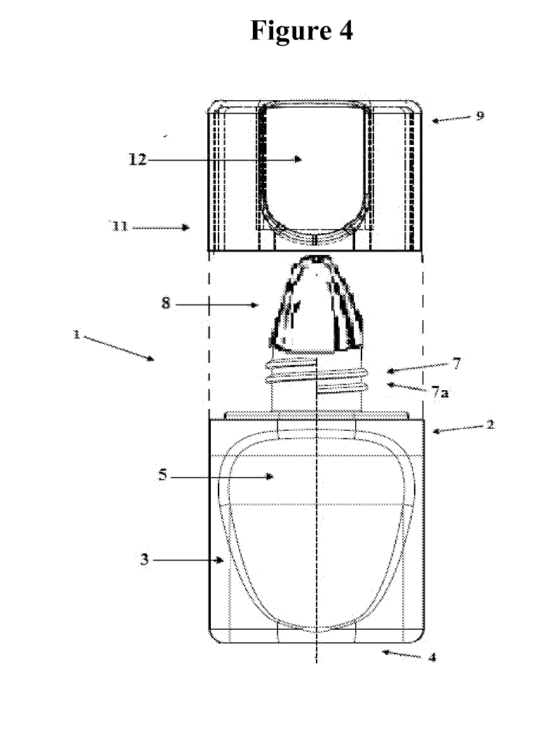

FIG. 4 provides a view of the finger indent 5 of the container, with the lid 9 showing the finger indent 12 on outer wall 11.

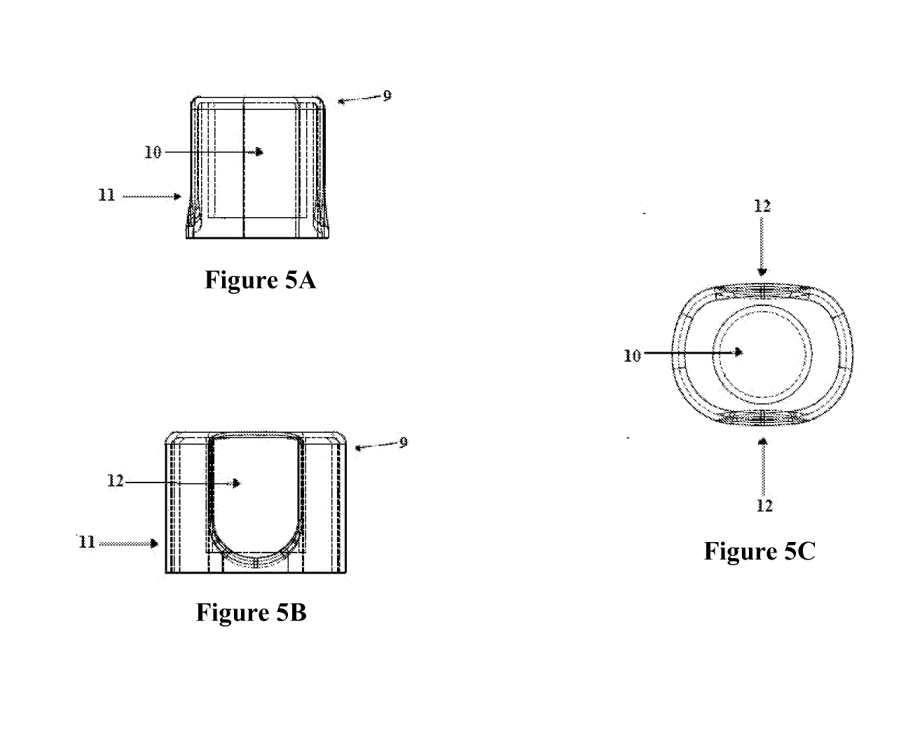

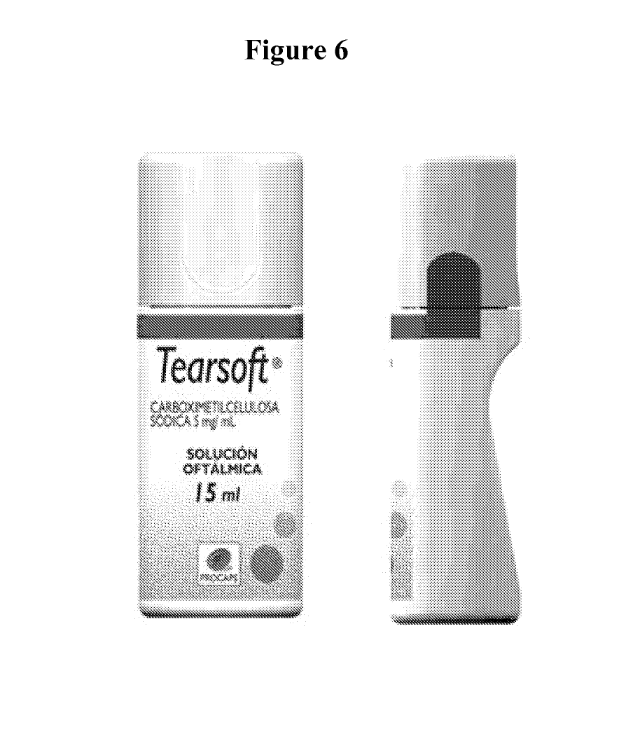

FIG. 5A is the lid 9 of the dispensing device showing tubular member 10 and outer wall 11.

FIG. 5B is a view of lid 9 of the invention showing the finger indent 12 in outer wall 11.

FIG. 5C is a top view of the lid of the invention showing tube 10 and finger indent 12.

The dispensing device of the invention is made of a thermoplastic material. Examples of the thermoplastic material serving as the structural material of the container unit 2 include polyethylene, polyethylene-polypropylene, polypropylene, polyethyethylene terephthalate, and polycarbonate and the like, and the overall container unit 2 is configured to allow elastic deformation.

The dispensing device of the invention is particularly useful for dispensing ophthalmic products to the eye of a mammal. Particularly useful products include olopatadine, dorzolamide, timolol, combinations of dorzolamide and timolol, latan oprost, quinolone antibiotics such as ciprofloxacin and levofloxacin, as well as all those ophthalmic drugs listed in the Merck Index and the Physicians Desk Reference of Drugs the contents of which are incorporated by reference herewith.

Products containing wetting agents such as carboxymethylcellulose in the dispensing device are also intended to be included in the instant invention.

The contents of all references cited in the instant specifications and all cited references in each of those references are incorporated in their entirety by reference herein as if those references were denoted in the text

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A container useful for making a dispensing device, said container having a resilient sidewall, a closed end, said resilient side wall including at least one finger indent, a threaded neck portion, and another resilient side wall opposite the resilient sidewall having at least one finger indent.

2. A dispenser comprising:
a container defining a reservoir, said container having a resilient sidewall, a closed end, and a dispensing tip opposite the closed end, said dispensing tip including a passageway in fluid communication with the reservoir, and said resilient side wall including at least one finger indent; and
a lid comprising a tube configured to cover the dispensing tip and an outer wall configured to align with the resilient side wall when the tube covers the dispensing tip, the outer wall including at least one finger indent;
wherein the lid is removably connectable to the container by a threaded connection between the tube and the dispensing tip.

3. The dispensing device of claim 2, further including an ophthalmic solution of an ophthalmic therapeutic agent.

4. The dispensing device of claim 3, wherein said ophthalmic therapeutic agent is carboxymethylcellulose.

5. The dispensing device of claim 3, wherein said ophthalmic therapeutic agent is olopatadine.

6. The dispensing device of claim 3, wherein said ophthalmic therapeutic agent is dorzolamide.

7. The dispensing device of claim 3, wherein said ophthalmic therapeutic agent is timolol.

8. The dispensing device of claim 3, wherein said ophthalmic therapeutic agent is a combination of dorzolamide and timolol.

9. The dispensing device of claim 3, wherein said ophthalmic therapeutic agent is latanoprost.

10. The dispensing device of claim 3, wherein said ophthalmic therapeutic agent is a quinolone antibiotic.

11. The dispensing device of claim 10, wherein said ophthalmic therapeutic agent is ciprofloxacin.

12. The dispensing device of claim 10, wherein said ophthalmic therapeutic agent is levofloxacin.
